(19) United States
(12) Patent Application Publication
(10) Pub. No.: US 2009/0155388 A1
(43) Pub. Date: Jun. 18, 2009

(54) WEIGHT PHYTO-NUTRACEUTICAL SYNERGISTIC COMPOSITION

(76) Inventor: Jose Angel Olalde, Clearwater, FL (US)

Correspondence Address: JOSE OLALDE RANGEL, 519 CLEVELAND STREET, SUITE 101, CLEARWATER, FL, 33755, US

(21) Appl. No.: 11/533,229

(22) Filed: Sep. 19, 2006

(51) Int. Cl. A61K 33/04; A61K 36/258; A61K 36/82; A61K 36/00

(52) U.S. Cl. 424/702; 424/728; 424/729; 424/725

(57) ABSTRACT

A Phytoceutical composition for the prevention and treatment of weight disorders and symptoms is provided. A specific combination of extracts of plants and nutraceuticals is provided, based on categorizing plants and nutraceuticals into one of three groups, Energy, Bio-Intelligence, and Organization. Such combinations have synergistic effects, with minimal side effects.

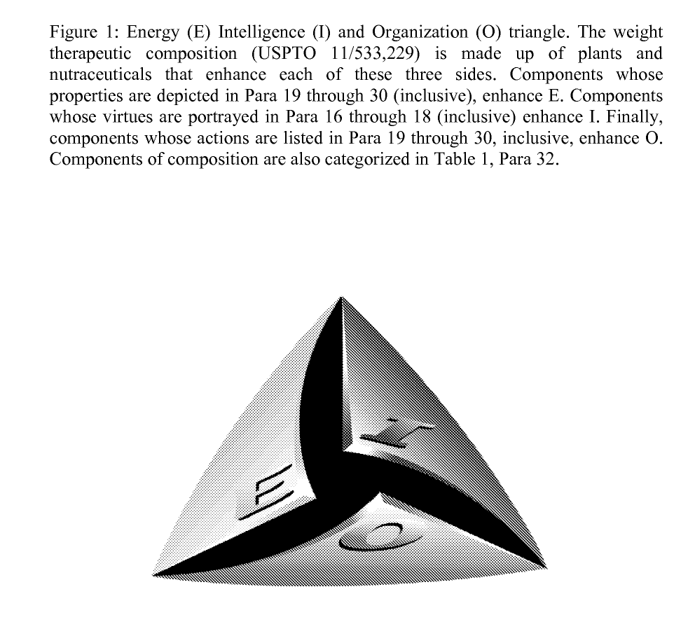

### PRIOR RELATED APPLICATIONS

Not applicable.

### FEDERALLY SPONSORED RESEARCH STATEMENT

Not applicable.

### REFERENCE TO MICROFICHE APPENDIX

Not applicable.

### FIELD OF THE INVENTION

The invention relates to a phytoceutical formulation used to treat weight disorders and symptoms. The formulation is a particular combination of plants that have synergistic effect in combination. Principles for selecting beneficial formulations are provided.

### BACKGROUND OF THE INVENTION

The academic study of medicinal plants for the treatment of diverse diseases has been nearly as pervasive as the study of Western medicines. The active principles from many traditional medicines have been extracted from plants, the curative agents identified and their mechanisms of action determined. Plant based medicines are typically well tolerated, with less severe side effects as well as a smaller range of side effects. In contrast, while synthetic drugs can be highly effective, their use is often hampered by severe side effects. Additionally, while synthetic pharmaceuticals are based upon single chemicals, many phytomedicines exert their beneficial effects through the additive or synergistic action of several chemical compounds acting at single or multiple target sites associated with a physiological process. As pointed out by Tyler (1999), this synergistic or additive pharmacological effect can be beneficial by eliminating the problematic side effects associated with the predominance of a single xenobiotic compound in the body.

In this respect, Kaufman et al. (1999) extensively documented how synergistic interactions underlie the effectiveness of a number of phytomedicines. This theme of multiple chemicals acting in an additive or synergistic manner likely has its origin in the functional role of secondary products in promoting plant survival. For example, in the role of secondary products as defense chemicals, a mixture of chemicals having additive or synergistic effects at multiple target sites would not only ensure effectiveness against a wide range of herbivores or pathogens but would also decrease the chances of these organisms developing resistance or adaptive responses (Kaufman et al., 1999; Wink, 1999). Conclusion: On one hand, synthetics may have the required efficacy for disease treatment; however this can be marred by severe side effects. On the other hand, despite the excellent medicinal qualities of many plants, they are individually insufficient to take chronic degenerative diseases into remission. However, there is mounting evidence which demonstrates that medical plants contain synergistic efficacy and/or side-effect neutralizing combinations (Gilani and Rahman, 2005). Thus, what are needed in the art are better treatment regimes with improved patient tolerance, while providing sufficient efficacy.

### SUMMARY OF THE INVENTION

A number of known beneficial plants and tonics were classified according to their capacity to enhance the three main elements that support overall health: Energy (E), Bio-intelligence (I) and Organization (O). A synergistic effect is expected when all three categories of herbs (E, I, O) are included in a formulation, preferably at least two or three or four plants from each category. Thus, an embodiment of the invention provides a method of selecting additional disease treating formulations according to these principles. Example of a formulation prepared this way is provided; additional formulations are being prepared and tested.

Another embodiment of the invention provides an effective, natural composition for treating excess weight and symptoms. The composition can be used alone, or can be combined with simultaneous use of one or more pharmaceutical compositions.

### DETAILED DESCRIPTION OF THE INVENTION

“Pharmaceutically acceptable excipients” is used herein according to art accepted meanings, and includes those ingredients needed to formulate a medicine for mammalian use, including the use of gelatin capsules.

“Synergistic” or “synergy” is used herein to mean that the effect is more than its additive property. In preferred embodiments, the synergy is at least 1.2, 1.5, 2, 5, or 10 fold.

By use of “plants,” what is meant herein is that the plant (or that portion with medicinal activity) is used whole, ground, or as an extract. Also included are purified active ingredients and derivatives thereof. However, it is believed that the best efficacy of plants used herein is achieved with the use of the entire plant or its extracts, rather than with the use of isolated active ingredients.

Further, although plants are named here according to commonly used nomenclature, with improving taxonomy plants are often reclassified. Whenever a plant is referenced, it includes related species with similar active ingredients.

The following examples are illustrative only and should not serve to unduly limit the invention.

### Example 1

### Plant Characteristics—Weight Control

### Energy Supplying Components.

Ajuga turkestanica: Its main active principle turkesterone, a phytoecdysteroid possessing an 11 alpha-hydroxyl group. Ecdysteroids normalize NADH dehydrogenase activity, enzyme which catalyzes electron transfer from NADH to ubiquinones in the oxidative phosphorylation processes which occur at the mitochondrial level, contributing to the potential electrochemical buildup required to produce ATP. It also normalizes the succinate dehydrogenase enzyme which participates in the tricarboxilic acid cycle, which translates to ATP synthesis and patient energy level increases [Tashmukhamedova M A, Almatov K T, Syrov V N. Comparative study of the effect of ecdysterone, turkesterone and nerobol on the function of rat liver mitochondria in experimental diabetes. Vopr Med. Khim. 1986; 32:24-8].

Panax ginseng (Chinese ginseng, Panax, ren shen, jintsam, ninjin, Asiatic ginseng, Japanese ginseng, Oriental ginseng, Korean red ginseng) The main active components are ginsenosides (protopanaxadiols and protopanaxatriols types) these have been shown to have a variety of beneficial effects, including anti-inflammatory, antioxidant, and anticancer effects. Energizing effect: Ginseng's active principles bind to the cellular membrane's beta-adrenergic receptors which unleashes the second messenger transduction routes (cyclic AMP). Subsequently the signal is transduced to the mitochondria to increase malate-dehydrogenase, succinate dehydrogenase and citrate synthetase activity. This increases ATP generation thus increasing the patient's energy levels. Crude saponin of Korean ginseng administration reduced body weight, food intake, and fat content. The hypothalamic NPY expression and serum leptin level were reduced after treatment. The results of this suggest that Korean ginseng may be useful in the treatment of obesity and related disorders as anti-obesity agents. (Kim J H, Hahm D H, Yang D C. Effect of crude saponin of Korean red ginseng on high-fat diet-induced obesity in the rat. J Pharmacol Sci. 2005; 97:124-31). Incorporation of Panax provides at least 86 active principles in a single therapeutic.

Rhodiola rosea (Golden Root, Roseroot, Artic root) consists mainly of phenylpropanoids (rosavin, rosin, rosarin—all specific to R. rosea), phenylethanol derivatives (salidroside, rhodioloside, tyrosol), flavanoids (catechins, proanthocyanidines, rodiolin, rodionin, rodiosin, acetylrodalgin, tricin), monoterpenes (rosiridol, rosaridin), fitosterols (daucosterol, beta-sitosterol), and phenolic acids (chlorogenic, caffeic, hydroxycinnamic and gallic acid). There are many species of Rhodiola, but rosavins seem to be unique to R. rosea, and it's the preferred species for this formulation. Rhodiola increases energy levels because it activates ATP synthesis and re-synthesis in mitochondria, stimulating reparative processes (Abidov M, Crendal F, Grachev S. Effect of extracts from R. rosea and R. crenulata(Crassulaceae) roots on ATP content in mitochondria of skeletal muscles. Bull Exp Biol Med. 2003; 136:585-7). Plant adaptogens are compounds that increase the ability of an organism to adapt to environmental factors and avoid damage from such factors. The beneficial effects of multi-dose administration of adaptogens are mainly associated with the hypothalamic-pituitary-adrenal (HPA) axis, a part of the stress-system that is believed to play a primary role in the reactions of the body to repeated stress and adaptation. In contrast, the single dose application of adaptogens is important in situations that require a rapid response to tension or to a stressful situation. In this case, the effects of the adaptogens are associated with another part of the stress-system, namely, the sympatho-adrenal-system (SAS) that provides a rapid response mechanism mainly to control the acute reaction of the organism to a stressor. Rhodiola rosea effectively increases mental performance and physical working capacity in humans. R. rosea is the most active of the adaptogens producing, within 30 min of administration, a stimulating effect that continues for at least 4-6 h. (Panossian A, Wagner H. Stimulating effect of adaptogens: an overview with particular reference to their efficacy following single dose administration. Phytother Res. 2005; 19:819-38). This plant promotes pyruvate participation in metabolic processes (Pogorelyi V E, Makarova L M. Rhodiola rosea extract for prophylaxis of ischemic cerebral circulation disorder. Eksp Klin Farmakol. 2002; 65:19-22). A clinical, randomized, controlled trial showed that Rhodiola rosea produces a statistically significant improvement in total mental performance, overall level of mental fatigue, involving complex perceptive and cognitive cerebral functions, such as associative thinking, short-term memory, calculation and ability of concentration, and speed of audio-visual perception (Darbinyan V, Kteyan A, Panossian A. Rhodiola rosea in stress induced fatigue—a double blind cross-over study of a standardized extract SHR-5 with a repeated low-dose regimen on the mental performance of healthy physicians during night duty. Phytomedicine. 2000; 7:365-71). A clinical, randomized, controlled trial showed that Rhodiola rosea produces a significant improvement in physical fitness, mental fatigue, neuro-motor tests and general well-being (Spasov A A, Wikman G K, Mandrikov V B. A double-blind, placebo-controlled pilot study of the stimulating and adaptogenic effect of Rhodiola rosea SHR-5 extract on the fatigue of students caused by stress during an examination period with a repeated low-dose regimen. Phytomedicine. 2000; 7:85-9). This phytomedicine provides at least 28 active principles in a single therapeutic.

Bio-Intelligence modulators.

Aralia mandshurica (Manchurian Thorn Tree) The main active principles are triterpene saponins aralosides (elatosides). Siberians traditionally preferred Aralia for immune health, to reduce stress/depression, and to improve physical and mental performance. Siberians would often combine Aralia with other adaptogens for maximal stress reduction/performance enhancement benefits. Aralia extract was included in the official Russian Pharmacopoeia in 1983 as a treatment for the symptoms of stress overload, such as fatigue, weakness, headache, libido loss, depression, immune weakness, etc. G. P. Gubina reported in 1988 a 90 percent success rate—using Aralia—in 106 patients treated for various “asthenic” (stress overload/weakness) conditions. Aralia enhances a person's ability for memorization and prolonged concentration. In proofreading tests, after taking this plant, a decrease in the quantity of mistakes was observed in 88 percent of the experimental group, while an increase in the quantity of mistakes was observed in 54 percent of the control group. Those taking Aralia mandshurica exerted a strong stimulating influence among test subjects who displayed a great improvement in reading comprehension, aptitude and speed. [A. A. Lebedev/Far East Scientific Center of the USSR]; [V. V. Kazakevich/Academy of Sciences, Vladivostok, Russia]. The effects of oral treatment with a phytopreparation containing Aralia mandshurica (Araliaceae) and Engelhardtia chrysolepis (Juglandaceae) extracts on some parameters of lipid metabolism was studied in 32 women with non-diabetic obesity receiving low-caloric diet. Our randomized placebo-controlled study showed that this treatment led to a decrease in total body weight and fat weight, reduced perilipin content in adipocytes and plasma triglyceride content, and stimulated activity of hormone-sensitive lipase (Abidov, M.; Rio, M.; Ramazanov, T. Effects of Aralia mandshurica and Engelhardtia chrysolepis extracts on some parameters of lipid metabolism in women with nondiabetic obesity. Bull Exp Biol Med. 2006; 141: 343-346).

Garcinia cambogia: (Malabar tamarind). Garcinia cambogia is a unique source of (−)-hydroxycitric acid (HCA), which exhibits a distinct sour taste and has been safely used for centuries in Southeastern Asia to make foods more filling and satisfying. The active principle constituent is (−)-Hydroxycitric acid (HCA).

Also, Garcinia has polyisoprenylated benzophenones, such as xanthochymol and isoxanthochymol and flavonoids. Extensive experimental studies show that HCA inhibits fat synthesis and reduces food intake. The primary mechanisms of action of HCA appears to be related to its ability to act as a competitive inhibitor of the extramitochondrial enzyme ATP-citrate lyase, which plays an essential role in de novo lipogenesis. Also, HCA inhibits adipogenic differentiation of preadipocytes, promotes fat oxidation, enhances serotonin release and availability in the brain cortex (which induces satiety and has a role in the normal termination of feeding), normalizes lipid profiles, and lowers serum insulin and leptin levels in obese subjects. A clinical randomized double-blind, placebo-controlled parallel trial in 89 mildly overweight women showed that the group treated with (−)-Hydroxycitric acid (HCA) from Garcinia cambogia achieved a significantly greater reduction of body weight. (Mattes R D, Bormann L. Effects of (−)-hydroxycitric acid on appetitive variables. Physiol Behav. 2000; 71:87-94). The study found that Garcinia extract inhibits the cytoplasmic lipid accumulation as well as adipogenic differentiation of preadipocytes. The mechanisms that regulate the inhibition of insulin-induced differentiation by Garcinia extracts include the inhibition of expression of the early adipogenic transcription factor, CCAAT element binding protein (C/EBP)alpha that regulate adipogenesis. These results suggest that the specific targets of Garcinia extract on differentiation process of 3T3-L1 cells could be, at least, early adipogenic differentiation factor (Kim M S, Kim J K, Kwon D Y. Anti-adipogenic effects of Garcinia extract on the lipid droplet accumulation and the expression of transcription factor. Biofactors. 2004; 22:193-6). HCA from Garcinia cambogia causes a significant decrease in body weight and reduction in feed consumption without any adverse effects (Soni M G, Burdock G A, Preuss H G. Safety assessment of (−)-hydroxycitric acid and Super CitriMax, a novel calcium/potassium salt. Food Chem Toxicol. 2004; 42:1513-29). HCA promotes fat oxidation, enhances serotonin release and availability in the brain cortex, normalizes lipid profiles, and lowers serum leptin levels in obese subjects. HCA has demonstrated to be conditionally effective in weight management in experimental animals as well as in humans.
HCA significantly contained body weight growth, a response associated with lowered abdominal fat leptin expression. Microarray analysis of 9960 genes present in the fat tissue identified a small set of specific genes sensitive to dietary HCA. Under the current experimental conditions, HCA proved to be effective in restricting body weight gain. Functional characterization of HCA-sensitive genes revealed that upregulation of genes encoding serotonin receptors represent a distinct effect of dietary HCA supplementation (Roy S, Rink C, Khanna S. Body weight and abdominal fat gene expression profile in response to a novel hydroxycitric acid-based dietary supplement. Gene Expr. 2004; 11:251-62). HCA supplementation demonstrated a reduction in body weight as compared to the corresponding controls (Shara M, Ohia S E, Yasmin T. Dose- and time-dependent effects of a novel (−)-hydroxycitric acid extract on body weight, hepatic and testicular lipid peroxidation, DNA fragmentation and histopathological data over a period of 90 days. Mol Cell Biochem. 2003; 254:339-46). Garcinia cambogia extract lowered the levels of serum insulin and leptin, as well as the leptin/WAT ratio. These findings suggested that G. cambogia extract efficiently improved glucose metabolism and displayed leptin-like activity (Hayamizu K, Hirakawa H, Oikawa D. Effect of Garcinia cambogia extract on serum leptin and insulin in mice. Fitoterapia. 2003; 74:267-73). Flavonoids from Garcinia cambogia exerted hypolipidaemic activity. A dose response study revealed biphasic activity. Higher doses were less effective in reducing lipid levels in serum and tissues, although devoid of toxic effects (Koshy A S, Vijayalakshmi N R. Impact of certain flavonoids on lipid profiles—potential action of Garcinia cambogia flavonoids. Phytother Res. 2001; 15:395-400). Garcinia cambogia inhibited the accumulation of lipid droplets and the peak droplet area shifted to become smaller. The activities of glycerophosphate dehydrogenase, a marker of adipose differentiation, were not significantly inhibited by the Garcinia extract. These findings suggest that the Garcinia extract inhibits lipid droplet accumulation in fat cells without affecting adipose conversion (Hasegawa N. Garcinia extract inhibits lipid droplet accumulation without affecting adipose conversion in 3T3-L1 cells. Phytother Res. 2001; 15:172-3).

Hoodia gordonii: (Ghaap, Hoodia “cactus”, South African “desert cactus”). The San Bushmen of the Kalahari, one of the world's oldest and most primitive tribes, had been eating the Hoodia for thousands of years, to stave off hunger during long hunting trips. Due to the tradition of food use of Hoodia plants, certain species were included in a scientific research project established by the South African statutory council known as CSIR (Council for Scientific and Industrial Research) to screen a large number of bush foods. The plant contains a previously unknown molecule, a oxypregnane steroidal glycoside with anorectic activity in animals, termed P57AS3 (P57). Hoodia gordonii is traditionally used in South Africa for its appetite suppressant properties. P57AS3 (P57), is the reported active constituent from this plant as an appetite suppressant (Avula B, Wang Y H, Pawar R S. Determination of the appetite suppressant P57 in Hoodia gordonii plant extracts and dietary supplements by liquid chromatography/electrospray ionization mass spectrometry (LC-MSD-TOF) and LC-UV methods. J AOAC Int. 2006; 89:606-11). Intracerebroventricular (i.c.v.) injections of the purified P57AS3 demonstrated that the compound has a likely central (CNS) mechanism of action. The studies demonstrated that the compound increases the content of ATP by 50-150% in hypothalamic neurons. In addition, third ventricle (i.c.v.) administration of P57, which reduces subsequent 24-h food intake by 40-60%, also increases ATP content in hypothalamus. With growing evidence of metabolic or nutrient-sensing by the hypothalamus, ATP may be a common currency of energy sensing, which in turn may trigger the appropriate neural, endocrine and appetitive responses as similar to other fundamental hypothalamic homeostatic centers for temperature and osmolarity (MacLean D B, Luo L G. Increased ATP content/production in the hypothalamus may be a signal for energy-sensing of satiety: studies of the anorectic mechanism of a plant steroidal glycoside. Brain Res. 2004; 1020:1-11). In 2001 Phytopharm completed a double-blind, placebo-controlled clinical study in overweight, but otherwise healthy volunteers using an extract of Hoodia gordonii. The large doses of extract caused a statistically significant reduction in the average daily calorie intake. In addition, a statistically significant reduction in body fat content was also observed compared to the placebo group after two weeks.

### Organizational Improvers.

Camellia sinensis (Tea): The polyphenols found in tea are more commonly known as flavanols or catechins. The main catechins in green tea are epicatechin, epicatechin-3-gallate, epigallocatechin, and epigallocatechin-3-gallate (EGCG), the latter being the highest in concentration. Tea also contains caffeine. Of the flavonols present in green tea, two in particular, (−)-epigallocatechin 3-O-gallate and (−)-epigallocatechin, have been shown to have significant activity. Action mechanisms: 1) Stimulation of thermogenesis, increased metabolic capacity and utilization of fatty acids. 2) Down-regulation of fatty acid synthase gene expression, which reduces the action of this enzyme, increases lipogenesis and stimulates cell energy expenditure. 3) Induction of apoptosis of adipose tissue 4) Pancreatic and gastric lipase inhibition. The compound (−)-epigallocatechin-3-gallate (EGCG) is the major catechin found in green tea [Camellia sinensis L. Ktze. (Theaceae)]. This polyphenolic compound and several related catechins are believed to be responsible for the potential health benefits ascribed to green tea and EGCG include enhancing weight loss. Well-designed double-blinded controlled clinical studies have recently demonstrated the efficacy of green tea extracts and purified EGCG products in patients (Nagle D G, Ferreira D, Zhou Y D. Epigallocatechin-3-gallate (EGCG): Chemical and biomedical perspectives. Phytochemistry. 2006; 67:1849-55). Recent human studies suggest that green tea may contribute to the promotion of physiological functions such as body weight control (Cabrera C, Artacho R, Gimenez R. Beneficial effects of green tea—a review. J Am Coll Nutr. 2006; 25:79-99). A series of polyphenols known as catechins are abundant in green tea. The effects of catechin-rich green tea extract were studied in one trial on mice. Running times to exhaustion were 30% higher and were accompanied by a lower respiratory exchange ratio, higher muscle beta-oxidation activity, and lower malonyl-CoA content. In addition, muscle glycogen content was higher and plasma lactate concentrations were significantly lower after exercise. The endurance-improving effects of catechins of green tea were mediated, at least partly, by increased metabolic capacity and utilization of fatty acid as a source of energy in skeletal muscle during exercise (Murase T, Haramizu S, Shimotoyodome A. Green tea extract improves running endurance in mice by stimulating lipid utilization during exercise. Am J Physiol Regul Integr Comp Physiol. 2006; 290:R1550-6). Among the health-promoting effects of tea and tea polyphenols the hypolipidemic and anti-obesity effects in animals and humans it has been demonstrated that the body weights of rats and their plasma triglyceride, cholesterol, and LDL-cholesterol have been significantly reduced by feedings of oolong, black, pu-erh, and green tea leaves. It has been suggested that increased lipogenesis may be through down-regulation of fatty acid synthase gene expression in the nucleus and stimulation of cell energy expenditure in the mitochondria. Experimental data suggests that the molecular mechanisms of fatty acid synthase gene suppression by tea polyphenols (EGCG, theaflavins) may invite down-regulation of EGFR/P13K/Akt/Sp-1 signal transduction pathways. Adipocyte apoptosis has been induced in vitro using (−)-epigallocatechin gallate (EGCG) from Camellia sinensis. Natural products have potential for inducing apoptosis of adipose tissue, inhibiting bone marrow adipogenesis, thereby yielding effective treatments for obesity (Nelson-Dooley C, Della-Fera M A, Hamrick M. Novel treatments for obesity and osteoporosis: targeting apoptotic pathways in adipocytes. Curr Med. Chem. 2005; 12:2215-25). Recent studies have shown that FAS (fatty acid synthase) is a potential therapeutic target of obesity. Camellia sinensis inhibits FAS effectively. The ability of GTE (green tea extract) to inhibit FAS is more potent than that of two known inhibitors in green tea leaves, EGCG (epigallocatechin gallate) and ECG (epicatechin gallate). (−)-CG (catechin gallate) is a very potent inhibitor of FAS, and may contribute to the high inhibitory effect of GTE on FAS (Zhang R, Xiao W, Wang X. Novel inhibitors of fatty-acid synthase from green tea (Camellia sinensis Xihu Longjing) with high activity and a new reacting site. Biotechnol Appl Biochem. 2006; 43:1-7). Camellia sinensis leaves contain 423 active principles.

Chromium picolinate: The element chromium apparently has a role in maintaining proper carbohydrate and lipid metabolism in mammals. As this role probably involves potentiation of insulin signaling, chromium dietary supplementation has been postulated to potentially have effects on body composition, including reducing fat mass and increasing lean body mass.

Because the supplement is absorbed better than dietary chromium, most studies have focused on the use of chromium picolinate [Cr(pic)(3)]. Human studies suggest that chromium picolinate (CrPic) decreases insulin levels and improves glucose disposal in obese and type 2 diabetic populations. A metanalysis on 10 randomized, double-blind and placebo-controlled studies showed a relatively small effect of chromium picolinate compared with placebo for reducing body weight (Pittler M H, Stevinson C, Ernst E. Chromium picolinate for reducing body weight: meta-analysis of randomized trials. Int J Obes Relat Metab Disord. 2003; 27:522-9). A clinical randomized controlled trial on 36 obese non-diabetic patients showed that Chromium picolinate is able to increase lean body mass in obese patients in the maintenance period after a very-low-calorie diet without counteracting the weight loss achieved (Bahadori B, Wallner S, Schneider H. Effect of chromium yeast and chromium picolinate on body composition of obese, non-diabetic patients during and after a formula diet. Acta Med Austriaca. 1997; 24:185-7). A clinical, randomized double-blind study in 37 patients with type 2 diabetes showed that CrPic supplementation in subjects with type 2 diabetes who are taking sulfonylurea agents significantly improves insulin sensitivity. It also improved glucose control and significantly attenuated body weight gain and visceral fat accumulation compared with the placebo group (Martin J, Wang Z Q, Zhang X H. Chromium picolinate supplementation attenuates body weight gain and increases insulin sensitivity in subjects with type 2 diabetes. Diabetes Care. 2006; 29:1826-32).

Cola acuminata (Abata Cola, Cola vera, Cola nitida): Cola acuminata contain xanthines—the same type of alkaloids found in tea and coffee. Common among these xanthine derivatives are caffeine, theophylline and theobromine. These xanthines are known to stimulate gastric acid secretion and improve utilization of fatty acids as a fuel source. Thermogenic ingredients may be considered as functional agents that could help in preventing obesity (Tbu J O, Tyama A C, Ijije C T. The effect of cola acuminata and cola nitida on gastric acid secretion. Scand J Gastroenterol Suppl. 1986; 124:39-45). Cola acuminata contains at least 32 active principles in a single therapeutic.

Coleus forskohlii BRIQ (Lamiaceae)—Forskohl's Coleus. The labdane diterpene forskolin, derived from the root of the plant, is the primary constituent of clinical interest in Coleus forskohlii. It was discovered by Western scientists in 1974 and was initially referred to as coleonol. Since that time, as other coleonols and diterpenoids have been identified, the name was changed to forskolin. Forskolin is responsible for virtually all pharmacological activities attributed to Coleus forskohlii; extracts of this constituent have been used in nearly all existing studies. There is evidence, however, that other plant constituents, such as volatile oils and other diterpenoids and coleonols, may contribute to the pharmacological activity and absorption of forskolin. Detailed analysis reveals approximately 20 constituents in various parts of the plant, but forskolin and other coleonols are present only in the root portion. Forskolin's primary mode of action is to increase cyclic adenosine monophosphate (cAMP) and cAMP-mediated functions, via activation of the enzyme adenyl cyclase. Forskolin has been shown to increase cAMP formation in all eukaryotic cells. Forskolin's potentiation of cAMP in turn stimulates lipolysis in adipocytes. A randomized, double-blind, placebo-controlled study in 30 overweight and obese men showed that Forskolin significantly decreased body fat percentage and fat mass compared with the placebo group. Also, there was a trend toward a significant increase for lean body mass in the forskolin group compared with the placebo group. The results indicate that forskolin is a possible therapeutic agent for the management and treatment of obesity (Godard M P, Johnson B A, Richmond S R. Body composition and hormonal adaptations associated with forskolin consumption in overweight and obese men. Obes Res. 2005; 13:1335-43). Coleus f. provides at least 20 active principles in a single therapeutic.

Engelhardtia chrysolepis (Cheo Tia): contains flavonoids and flavonoid-related compounds, such as dihydroquercetin 3-rhamnoside and dihydroflavonols such as taxifolin and its glycoside, astilbin (dihydroflavonol rhamnoside). The effects of oral treatment with a phytopreparation containing Aralia mandshurica (Araliaceae) and Engelhardtia chrysolepis (Juglandaceae) extracts on some parameters of lipid metabolism was studied in 32 women with nondiabetic obesity receiving low-caloric diet.

This randomized placebo-controlled study showed that this treatment led to a decrease in total body weight and fat weight, reduced perilipin content in adipocytes and plasma triglyceride content, and stimulated activity of hormone-sensitive lipase (Abidov, M.; Rio, M.; Ramazanov, T. Effects of Aralia mandshurica and Engelhardtia chrysolepis extracts on some parameters of lipid metabolism in women with nondiabetic obesity. Bull Exp Biol Med. 2006; 141:343-346). Astilbin, a dihydroflavonol rhamnoside isolated from the leaves of Engelhardtia chrysolepis, enhanced the release of lipoprotein lipase (LPL) activity. Astilbin alone in a dose-dependent manner stimulated Lipolysis. Astilbin may enhance the release of LPL activity through a synergistic effect on an increase in the cellular cAMP content produced by vanadate accompanied by more potent activation of cAMP-dependent protein kinase (Motoyashiki T, Miyake M, Morita T. Enhancement of the vanadate-stimulated release of lipoprotein lipase activity by astilbin from the leaves of Engelhardtia chrysolepis. Biol Pharm Bull. 1998; 21:517-9).

Gymnema sylvestre: Extracts of this plant are widely used in Australian, Japanese, Vietnamese and Indian folk medicine. Gymnema s. contains gurmarin (polypeptide), gymnemagenin (saponin), gymnemic acids (triterpene glycosides), and gymnemate. A clinical randomized controlled trial in 90 obese subjects showed that the group that received a combination of hydroxycitric acid, niacin-bound chromium and Gymnema sylvestre lowered body weight, BMI decreased as well, low-density lipoprotein and triglycerides levels were reduced, while high-density lipoprotein levels increased, serum leptin levels decreased, serotonin levels increased and urinary excretion of fat metabolites increased, to a greater extent than the other groups. Conclusions: the combination of HCA-SX plus NBC and GSE reduce body weight and BMI, suppress appetite, improve blood lipid profiles, increase serum leptin and serotonin levels and increase fat oxidation more than placebo (Preuss H G, Garis R I, Bramble J D. Efficacy of a novel calcium/potassium salt of (−)-hydroxycitric acid in weight control. Int J Clin Pharmacol Res. 2005; 25:133-44). Supplementation with gymnemate extracted from Gymnema sylvestre promoted weight loss by its ability to reduce hyperlipidemia, which was no withdrawal rebound.

Supplementation with gymnemate is a novel therapeutic tool for weight management. (Luo H, Kashiwagi A, Shibahara T. Decreased bodyweight without rebound and regulated lipoprotein metabolism by gymnemate in genetic multifactor syndrome animal. Mol Cell Biochem. 2006, May 12; Epub). OB-200G is a polyherbal preparation containing aqueous extracts of Garcinia cambogia, Gymnema sylvestre, Zingiber officinale, Piper longum and resin from Commiphora mukul, all possessing thermogenic properties. Our previous studies reveal OB-200G to exert antiobesity effects in dietary animal models of obesity. The hyperphagic effect of a 5-HT depletor, a 5-HT1A agonist, a 5-HT antagonist and a glucose antimetabolite was significantly antagonized by both OB-200G and fluoxetine. Seganserin, a centrally acting 5-HT2 antagonist, markedly attenuated the satiety action of OB-200G. The present observations suggest the role of serotonin in mediation of satiety by OB-200G and hence its antiobesity effect (Kaur G, Kulkarni S K. Investigations on possible serotonergic involvement in effects of OB-200G (polyherbal preparation) on food intake in female mice. Eur J. Nutr. 2001; 40:127-33). Gymnema sylvestre suppressed body weight gain and accumulation of liver lipids. In addition, intraperitoneal fat and fat drop vacuoles on the epithelium of renal tubules, were scattered. Also, plasma triglyceride levels decreased. (Shigematsu N, Asano R, Shimosaka M. Effect of long term-administration with Gymnema sylvestre R. BR on plasma and liver lipid in rats. Biol Pharm Bull. 2001; 24:643-9). Gymnemic acid from Gymnema sylvestre, is known to inhibit the intestinal absorption of glucose in humans and rats. Gymnemic acid potently inhibits the intestinal absorption of oleic acid. Gymnema preparations have a profound action on the modulation of taste, particularly suppressing sweet taste sensations. It is used in the treatment of diabetes mellitus and against obesity and caries. Lipid lowering and other effects are also reported (Porchezhian E, Dobriyal R M. An overview on the advances of Gymnema sylvestre: chemistry, pharmacology and patents. Pharmazie. 2003; 58:5-12).

Gynostemma pentaphyllum: Contains Gypenosides (triterpenoid saponins); dammarane glycosides, gylongiposide I, allantion, vitexin, Gynosides A-E (ocotillone-type saponins); rutin, ombuoside and malonic acid (flavonoids). Other than weight reduction by dieting or physical activity, there are no well-documented medical treatments for fatty liver disease. A randomized, single-blind, controlled clinical trial showed the efficacy of the add-on Gynostemma pentaphyllum in 56 subjects with nonalcoholic fatty liver disease. Body Mass Index, aspartate aminotransferase (AST), alkaline phosphatase, insulin and insulin resistance index (HOMA-IR), uric acid and fatty liver score were significantly reduced. CONCLUSION: GP is an effective adjunct treatment to diet therapy for patients with nonalcoholic fatty liver disease (Chou S C, Chen K W, Hwang J S. The add-on effects of Gynostemma pentaphyllum on nonalcoholic fatty liver disease. Altern Ther Health Med. 2006; 12:34-9). Gynostemma pentaphyllum is widely used in traditional Chinese medicine. Preliminary studies indicate Gynostemma lower cholesterol. Oral administration of gypenoside extract reduced triglyceride and total cholesterol levels. These studies demonstrate efficacy of Gynostemma pentaphyllum in lowering triglyceride and cholesterol in acute hyperlipidemia (Megalli S, Aktan F, Davies N M. Phytopreventative anti-hyperlipidemic effects of gynostemma pentaphyllum in rats. J Pharm Pharm Sci. 2005 16; 8:507-15). Gypenosides of Gynostemma pentaphyllum inhibit lipid peroxidation in vascular endothelial cells. It also protected biomembranes from oxidative injury by reversing the decreased membrane fluidity of mitochondria, increasing mitochondrial enzyme activity in vascular endothelial cells and decreasing intracellular lactate dehydrogenase leakage from these cells. The extensive antioxidant effect of GP is of value to the prevention and treatment of various diseases such as atherosclerosis. (Li L, Jiao L, Lau B H. Protective effect of gypenosides against oxidative stress in phagocytes, vascular endothelial cells and liver microsomes. Cancer Biother. 1993; 8:263-72).

Hydrocotile asiatica (Gotu Kola, Bramhi, Pennywort, Marsh Penny, Pennywort and Centella asiatica) contains terpenoids (asiaticoside, brahmoside and brahminoside), aglycones (saponin glycosides), asiaticentoic acid, centellic acid, centoic acid and madecassic acid, sesquiterpenes (caryophyllene, trans-B-farnesene), volatile oils (Germacrene D), alkaloids (hydrocotylin), flavonoids (Quercetin, kaempferol), phytosterols (stigmasterol and sitosterol), and vallerine, fatty acids, resin, and tannins. Gotu Kola has been used for centuries in Ayurvedic and traditional Chinese medicine to alleviate symptoms of anxiety.

Also, Gotu kola offers a lipolytic effect. A number of plants with purported anxiolytic activity bind to cholecystokinin (CCK) receptors. CCK is involved in the pathophysiology of anxiety. This double-blind, randomized clinical placebo-controlled study showed the anxiolytic activity of Gotu Kola (Centella asiatica) in healthy subjects (Bradwejn J, Zhou Y, Koszycki D. A double-blind, placebo-controlled study on the effects of Gotu Kola (Centella asiatica) on acoustic startle response in healthy subjects. J Clin Psychopharmacol. 2000; 20:680-4). A clinical controlled trial showed that slimming liposomes containing two micro-circulation activators, i.e., esculoside and Centella asiatica extracts, one phosphodiesterase inhibitor, i.e., caffeine, and one fatty acid-beta oxidation activator, i.e., L-carnitine induced a dramatic increase in the cyclic adenosine monophosphate (cAMP) content in human adipocytes, with a subsequent rise in the nonesterified fatty acids (NEFA) content of human adipocyte. It also, showed that slimming liposomes could provide an actual potent slimming effect on human volunteers. Slimming liposomes were able to antagonize the alpha(2)-adrenergic receptor that is known to reduce intracellular AMPc content and, subsequently, to down-regulate lipolysis (Tholon L, Neliat G, Chesne C, Saboureau D. An in vitro, ex vivo, and in vivo demonstration of the lipolytic effect of slimming liposomes: An unexpected alpha(2)-adrenergic antagonism. J Cosmet Sci. 2002; 53:209-18). Centella provides 59 active principles in a single therapeutic.

Ilex paraguariensis (Mate, Paraguay Tea, South American Holly): Contains xanthines—the same type of alkaloids found in tea and coffee. Common among these xanthine derivatives are theobromine, theophylline and caffeine. Also, nicotinic-acid which is lipolytic and ursolic-acid which acts as an antihyperlipidemic. ‘YGD’ a mixed herbal preparation is containing Yerba Mate (leaves of Ilex paraguayenis), Guarana (seeds of Paullinia cupana) and Damiana (leaves of Tumera diffusa var. aphrodisiaca) was studied in a double-blind placebo-controlled parallel trial. YGD significantly prolonged gastric emptying time and achieved body weight reductions. Active treatment with YGD capsules resulted in weight maintenance. YGD reduced the time to perceived gastric fullness and induced significant weight loss over 45 days in overweight patients treated in a primary health care context. Maintenance treatment given in an uncontrolled context resulted in no further weight loss, nor weight regain in the group as a whole. The herbal preparation is thus shown to be one that significantly modulates gastric emptying (Andersen T, Fogh J. Weight loss and delayed gastric emptying following a South American herbal preparation in overweight patients. J Hum Nutr Diet. 2001; 14:243-50). The results of a clinical randomized controlled trial showed that Ilex paraguariensis decreased the respiratory quotient, indicating a rise in the proportion of fat oxidized. The results suggested the potential of this plant in the treatment of obesity (Martinet A, Hostettmann K, Schutz Y. Thermogenic effects of commercially available plant preparations aimed at treating human obesity. Phytomedicine. 1999; 6:231-8). Ilex paraguariensis contains xanthines: theobromine, theophylline and caffeine. These xanthines are known to stimulate gastric acid secretion and improve utilization of fatty acids as a fuel source. Thermogenic ingredients may be considered as functional agents that could help in preventing obesity (Tbu J O, Tyama A C, Ijije C T. The effect of cola acuminata and cola nitida on gastric acid secretion. Scand J Gastroenterol Suppl. 1986; 124:39-45). Ilex contains no less than 33 active principles.

Paullinia cupana (Guarana): Contains xanthines—the same type of alkaloids found in tea and coffee. Common among these xanthine derivatives are theobromine, theophylline, caffeine, xanthine and hypoxanthine. These xanthines are known to stimulate gastric acid secretion and improve utilization of fatty acids as a fuel source. Thermogenic ingredients may be considered as functional agents that could help in preventing obesity. (Tbu J O, Tyama A C, Ijije C T. The effect of cola acuminata and cola nitida on gastric acid secretion. Scand J Gastroenterol Suppl. 1986; 124:39-45). Also, contains flavonoids ((+)-catechin, and (−)-epicatechin), cyanolipids, acylglycerols (oleic acid), cis-11-octadecenoic and cis-11-eicosenoic acids, paullinic acid, two methylbenzenes, one cyclic monoterpene, two cyclic sesquiterpene hydrocarbons, two methoxyphenylpropenes and two alkylphenol derivatives, estragole and anethole. ‘YGD’ a mixed herbal preparation is containing Yerba Mate (leaves of Ilex paraguayenis), Guarana (seeds of Paullinia cupana) and Damiana (leaves of Turnera diffusa var. aphrodisiaca) was studied in a double-blind placebo-controlled parallel trial.

YGD significantly prolonged gastric emptying time and achieved body weight reductions. Active treatment with YGD capsules resulted in weight maintenance. YGD reduced the time to perceived gastric fullness and induced significant weight loss over 45 days in overweight patients treated in a primary health care context. Maintenance treatment on an uncontrolled context resulted in no further weight loss, nor weight regain in the group as a whole. The herbal preparation, thus, significantly modulates gastric emptying (Andersen T, Fogh J. Weight loss and delayed gastric emptying following a South American herbal preparation in overweight patients. J Hum Nutr Diet. 2001; 14:243-50). The tonic action of Paullinia cupana decreased the liver glycogen contents of mice. Also, it significantly suppressed exercise-induced hypoglycemia. These findings indicate that the suppressive mechanism of hypoglycemia might be due to the promotion of glycogen resolution (Miura T, Tatara M, Nakamura K. Effect of guarana on exercise in normal and epinephrine-induced glycogenolytic mice. Biol Pharm Bull. 1998; 21:646-8). Mice that ingested a suspension of Paullinia cupana showed a significant increase in physical capacity when subjected to a stressful situation (Espinola E B, Dias R F, Mattei R. Pharmacological activity of Guarana (Paullinia cupana Mart.) in laboratory animals. J Ethnopharmacol. 1997; 55:223-9). This plant incorporates at least 19 active principles in a single therapeutic.

Selenium is a trace mineral that is essential to good health but required only in small amounts. Selenium is incorporated into proteins to make selenoproteins, which are important antioxidant enzymes. The antioxidant properties of selenoproteins help prevent cellular damage from free radicals. Other selenoproteins help regulate thyroid function and play a role in the immune system. There is evidence that selenium deficiency may contribute to development of a form of heart disease, hypothyroidism, and a weakened immune system. There is also evidence that selenium deficiency does not usually cause illness by itself. Rather, it can make the body more susceptible to illnesses caused by other nutritional, biochemical or infectious stresses. Gastrointestinal disorders may decrease the absorption of selenium, resulting in selenium depletion or deficiency.

Most cases of selenium depletion or deficiency are associated with severe gastrointestinal problems, such as Crohn's disease, or with surgical removal of part of the stomach. These and other gastrointestinal disorders can impair selenium absorption. People with acute severe illness who develop inflammation and widespread infection often have decreased levels of selenium. Alternatively, people dependent on food grown from selenium-deficient soil are also at risk of deficiency. People with iodine deficiency may also benefit from selenium supplementation. Researchers believe that selenium deficiency may worsen the effects of iodine deficiency on thyroid function, and that adequate selenium nutritional status may help protect against some of the effects of iodine deficiency. Researchers involved in the Supplementation en Vitamines et Mineraux AntioXydants (SU.VI.MAX) study in France, which was designed to assess the effect of vitamin and mineral supplements on chronic disease risk, evaluated the relationship between goiter and selenium in a subset of this research population. Their findings suggest that selenium supplements may be protective against goiter. A clinical study on healthy men fed foods naturally high or low in selenium for 120 days showed that by day 64, the low selenium group began to lose weight, and the weight changes were significantly different between groups (Hawkes W C, Keim N L. Dietary selenium intake modulates thyroid hormone and energy metabolism in men. J Nutr. 2003; 133:3443-8).

Tribulus terrestris (Caltrop, Yellow Vine, bindy eye, bindii, bullhead, burnut, burra gokhroo, caltrop, calthrops, cat's head, common dubbeltjie, devil's thorn, devil's weed, doublegee, dubbeltje, goathead, gokshura, ground bur-nut, isiHoho, land caltrop, Maltese cross, Mexican sandbur, puncture vine, puncture weed, rose, small caltrops, tackweed, Texas sandbur, yellow vine and Goathead). The fruits and roots of Tribulus contain active principles such as: phytosterols, flavonoids, alkaloids, glucosides and steroidal saponins of the furostanol sub-class. These active principles could significantly lower the levels of serum Total cholesterol, LDL-c and liver Total cholesterol, triglycerides, and increase the activities of superoxide-dismutase (SOD) in liver (Chu S, Qu W, Pang X. Effect of saponin from Tribulus terrestris on hyperlipidemia. Zhong Yao Cai. 2003; 26:341-4);

(Li M, Qu W, Chu S. Effect of the decoction of tribulus terrestris on mice gluconeogenesis. Zhong Yao Cai. 2001; 24:586-8). Tribulus provides at least 47 active principles in a single therapeutic.

### Example 2

### Composition—Weight Control

A particularly preferred composition is shown in Table 1. Ratios reflect the concentration of active ingredient over the natural state, and the amounts provided are mg of extract. Obviously, the amount should be increased where the strength is reduced, and vice versa.

A particularly preferred composition is shown in Table 1.

TABLE 1
Composition

| Active Agent | Ratio | Amount (mg) |
|---|---|---|
| Energy enhancers | | |
| Ajuga turkestanica | 5:1 | 65 |
| Panax ginseng | 1:1 | 255 |
| Rhodiola rosea | 5:1 | 26 |
| Bio-Intelligence modulators | | |
| Aralia mandshurica | 5:1 | 26 |
| Garcinia cambogia | 5:1 | 65 |
| Hoodia gordonii | 5:1 | 26 |
| Organization improvers | | |
| Camelia sinensis | 5:1 | 65 |
| Cola acuminata | 5:1 | 13 |
| Coleus forskohlii | 5:1 | 15 |
| Chromium picolinate | 1:1 | 0.052 |
| Engelhardtia chrysolepis | 5:1 | 26 |
| Gymnema sylvestre | 5:1 | 65 |
| Gynostemma pentaphyllum | 10:1 | 65 |
| Hydrocotile asiatica | 5:1 | 26 |
| Ilex paraguariensis | 5:1 | 26 |
| Paullinia cupana | 5:1 | 26 |
| Selenium | 1:1 | 45 |
| Tribulus terrestris | 5:1 | 65 |
| Total | | 900.052 |

### Example 3

### Weight Disorders Effectiveness and Tolerance—Study One

Patient response to this composition was examined through a 3 month long prospective, descriptive, multicenter study with 80 individuals with varying degrees of obesity (overweight). The administration of the composition significantly reduced patients weight in 83.75% of the patients. Only 3.75% (3 patients) of the study group observed mild secondary effects, which did not warrant the suspension of the treatment. The formula was considered an interesting alternative which with a combination of diet, exercise and other treatments may produce an unexpectedly superior therapeutic answer to this disorder.

### Example 4

### Principles for Selecting Synergistic Combinations

In order to explain the range of formulations encompassed by the invention, we have categorized beneficial plants and nutraceuticals into one of three groups, each of which should be present for synergistic effect. The classifications are: Energy, Bio-Intelligence and Organization. Plants and nutraceuticals classified under Energy are associated with ATP synthesis (such as the Krebs cycle, oxidative phosphorylation, beta-oxidation, etc.). Plants and nutraceuticals classified under Bio-Intelligence are those that regulate the neuroendocrine and immunological systems and cellular processes, thus controlling interactions between the various systems in the body. Finally, plants and nutraceuticals classified under Organization are those that relate to the structure and function of specific organs. Combinations of plants and nutraceuticals from these three classification groups have synergistic effect because they address each necessary component of cellular and organic health; providing the triangle—see Drawing # 1—on which healing is fully supported.

An illustrative example of synergy in medicinal plants is an in vitro study that demonstrates how the activity of herbal Berberine alkaloids is strongly potentiated by the action of 5′-methoxyhydnocarpin (5′-MHC)—an active principle of another phytomedicine (denominated Hydnocarpus wightiana).

It shows a strong increase of accumulation of berberine in the cells in the presence of 5′-MHC, indicating that this plant compound effectively disabled the bacterial resistance mechanism against the berberine antimicrobial, thus showing the synergy of both substances. Stermitz F R, et al., Synergy in a medicinal plant: antimicrobial action of berberine potentiated by 5′-methoxyhydnocarpin, a multidrug pump inhibitor. Proc Natl Acad Sci USA. 2000; 97:1433-7.

A further demonstration may be provided of synergistic effect on a molecular scale by studying the gene expression profile changes in response to various plant ingredients and combinations thereof. Experiments are already underway demonstrating the expression profile in response to the formulations. There is aid in this work because researchers have already begun studying the expression profiles of various medicinal plants, thus providing a database of knowledge from which to build. E.g., Gohil, et al., mRNA Expression Profile of a Human Cancer Cell Line in Response to Ginkgo Biloba Extract: Induction of Antioxidant Response and the Golgi System, Free Radic Res. 2001; 33:831-849.

Finally there may be further presentation of gene expression results using whole-genome microarray analysis to demonstrate the formulation's capability to provide gene activation (upregulation or downregulation).

What is claimed is:

1. A phytoceutical composition, comprising plants or extracts or active ingredients derived from each of the following plants and nutraceuticals: Ajuga, Panax, Rhodiola, Aralia, Garcinia, Hoodia, Camelia, Cola, Coleus, Chromium picolinate, Engelhardtia, Gymnema, Gynostemma, Hydrocotile, Ilex, Paullina, Selenium and Tribulus, together with pharmaceutically acceptable excipients.

2. The phytoceutical composition of claim 1, further comprising: Ajuga turkestanica, Panax ginseng, Rhodiola rosea, Aralia mandshurica, Garcinia cambogia, Hoodia gordonii, Camelia sinensis, Cola acuminata, Coleus forskohlii, Chromium picolinate, Engelhardtia chrysolepis, Gymnema sylvestre, Gynostemma pentaphyllum, Hydrocotile asiatica, Ilex paraguariensis, Paullina cupana, Selenium and Tribulus terrestris together with pharmaceutically acceptable excipients.

3. The phytoceutical composition of claim 2, comprising the relative amounts of ingredients shown in Table 1, and optionally including water or gelatin.

4. A method of treating disease comprising administering an effective amount of the composition of claim 3 to a patient sufficient to alleviate said disease.

5. The method of claim 4, wherein the disease are weight disorders and symptoms disorder.

6. A phytoceutical composition, comprising plants or extracts or active ingredients derived from each of the following plants and nutraceuticals: Ajuga turkestanica, Panax ginseng, Rhodiola rosea, Aralia mandshurica, Garcinia cambogia, Hoodia gordonii, Camelia sinensis, Cola acuminata, Coleus forskohlii, Chromium picolinate, Engelhardtia chrysolepis, Gymnema sylvestre, Gynostemma pentaphyllum, Hydrocotile asiatica, Ilex paraguariensis, Paullina cupana, Selenium and Tribulus terrestris; together with pharmaceutically acceptable excipients.

7. The phytoceutical composition of claim 1, whereby a particularly preferred composition comprises: Ajuga turkestanica 65 mg, Panax ginseng 255 mg, Rhodiola rosea 26 mg, Aralia mandshurica 26 mg, Garcinia cambogia 65 mg, Hoodia gordoni±26 mg, Camelia sinensis 65 mg, Cola acuminata 13 mg, Coleus forskohli±15 mg, Chromium picolinate 0.052 mg, Engelhardtia chrysolepis 26 mg, Gymnema sylvestre 65 mg, Gynostemma pentaphyllum 65 mg, Hydrocotile asiatica 26 mg, Ilex paraguariensis 26 mg, Paullina cupana 26 mg, Selenium 45 mg and Tribulus terrestris 65 mg; together with pharmaceutically acceptable excipients.

8. The phytoceutical composition of claim 7 and optionally including water or gelatin.

9. A method of treating disease comprising administering an effective amount of the composition of claim 8 to a patient sufficient to alleviate said disease.

10. The method of claim 9, wherein the disease are weight disorders and symptoms disorder.
